(19) United States
(12) Patent Application Publication
(10) Pub. No.: US 2014/0088523 A1
(43) Pub. Date: Mar. 27, 2014

(54) GARMENT FOR TREATMENT OF APPENDAGES

(71) Applicants: Patrick Thomas Ottuso, Vero Beach, FL (US); Keith Kalish, Vero Beach, FL (US)

(72) Inventors: Patrick Thomas Ottuso, Vero Beach, FL (US); Keith Kalish, Vero Beach, FL (US)

(21) Appl. No.: 13/949,339

(22) Filed: Jul. 24, 2013

Related U.S. Application Data
(60) Provisional application No. 61/705,768, filed on Sep. 26, 2012.

(51) Int. Cl. A61M 35/00; A43B 17/14

(52) U.S. Cl. CPC A61M 35/00; A43B 17/14
USPC 604/293; 2/69; 2/239

(57) ABSTRACT

A garment used for the treatment of appendages. The garment may include a plurality of polymer inserts with at least one protrusion. An ointment may be applied to the polymer inserts. A user may place the garment on an appendage and the plurality of polymer inserts may apply the ointments to an area to be treated.

### CROSS-REFERENCE TO RELATED APPLICATION

This application claims the benefit of priority of U.S. provisional application No. 61/705,768, filed Sep. 26, 2012, the contents of which are herein incorporated by reference.

### BACKGROUND OF THE INVENTION

The present invention relates to the treatment of damaged skin and, more particularly, to treatment of damaged skin using a garment with at least one polymer insert.

A wide variety of people have damaged and dry skin on their feet due to diseases such as xerosis, hyperkeratosis, fissuring, psoriasis, eczema and the like.

Generally, to relieve some of the symptoms, a person may apply a medical ointment or cream to ones foot. A convenient time to apply a medical ointment is during the night, when a person is laying dormant for hours. However, when a person applies an ointment for overnight use, the ointment may eventually rub off. Therefore, a user may spend most of the night without treating the disease.

As can be seen, there is a need for a device that secures and concentrates an ointment over problematic areas.

### SUMMARY OF THE INVENTION

In one aspect of the present invention, a medical apparel comprises: a garment formed to fit on an appendage of an animal, wherein the garment comprises an interior surface and an exterior surface; at least one polymer insert comprising an outer surface and an inner surface, wherein the inner surface of the polymer insert is adjacent to the interior surface of the garment; and at least one protrusion protruding from the outer surface of the polymer insert.

In another aspect of the present invention, a medical apparel comprising: a sock garment comprising an interior surface and an exterior surface, wherein the sock garment is formed to fit on a foot of a human; a plurality of polymer inserts comprising a lateral insert, a medial insert, and a heel insert, wherein the plurality of inserts is attached to the interior surface of the sock garment.

In another aspect of the present invention, a method of treating a foot comprises: providing a medical apparel comprising: a garment comprising an interior surface and an exterior surface; at least one polymer insert comprising an outer surface and an inner surface, wherein the inner surface of the polymer insert is adjacent to the interior surface of the garment; and at least one protrusion protruding from the outer surface of the polymer insert; applying an emollient to the outer surface of the at least one polymer insert; and placing the medical apparel on a foot to be treated.

These and other features, aspects and advantages of the present invention will become better understood with reference to the following drawings, description and claims.

### BRIEF DESCRIPTION OF THE DRAWINGS

FIG. 1 is a perspective view of the present invention shown in use;

FIG. 2 is a perspective view of the present invention;

FIG. 3 is a section detail view of the present invention along line 3-3 in FIG. 2;

FIG. 4 is a bottom view of the present invention shown in use; and

FIG. 5 is a section/cutaway view of the present invention along line 5-5 in FIG. 4.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a garment used for the treatment of appendages. The garment may include a plurality of polymer inserts with at least one protrusion. An ointment may be applied to the polymer inserts. A user may place the garment on an appendage and the plurality of polymer inserts may apply the ointments to an area to be treated.

In certain embodiments, the present invention may be used for treating disorders including, but not limited to, xerosis, hyperkeratosis, fissuring, psoriasis, eczema, or the like. The present invention may relieve dry or cracked skin, fissuring of the skin, and/or resolve clavi. The present invention may contain ointments for the skin. The ointments may include, but are not limited to, emollients and moisturizers. In certain embodiments, the ointments treat the diseases mentioned above.

In certain embodiments, the present invention may treat an appendage of an animal, such as an appendage of a human which may include, but is not limited to, the hand and/or the foot. When the device treats a foot, the device may cover the anterior, lateral, medial and posterior surfaces of the foot. In certain embodiments, the device is form fitted and sized to fit snuggly against the foot or any appendage to be treated. The device may be stretchable for easy application. In certain embodiments, the device is formed to easily slip on a foot, similar to putting on a shoe. Further, if made for a hand, the device may be similar to a glove. The device may include sizes for males and females. In certain embodiments, the device may be different colors and patterns, including but not limited to, black, blue, pink, beige, leopard print and camouflage print.

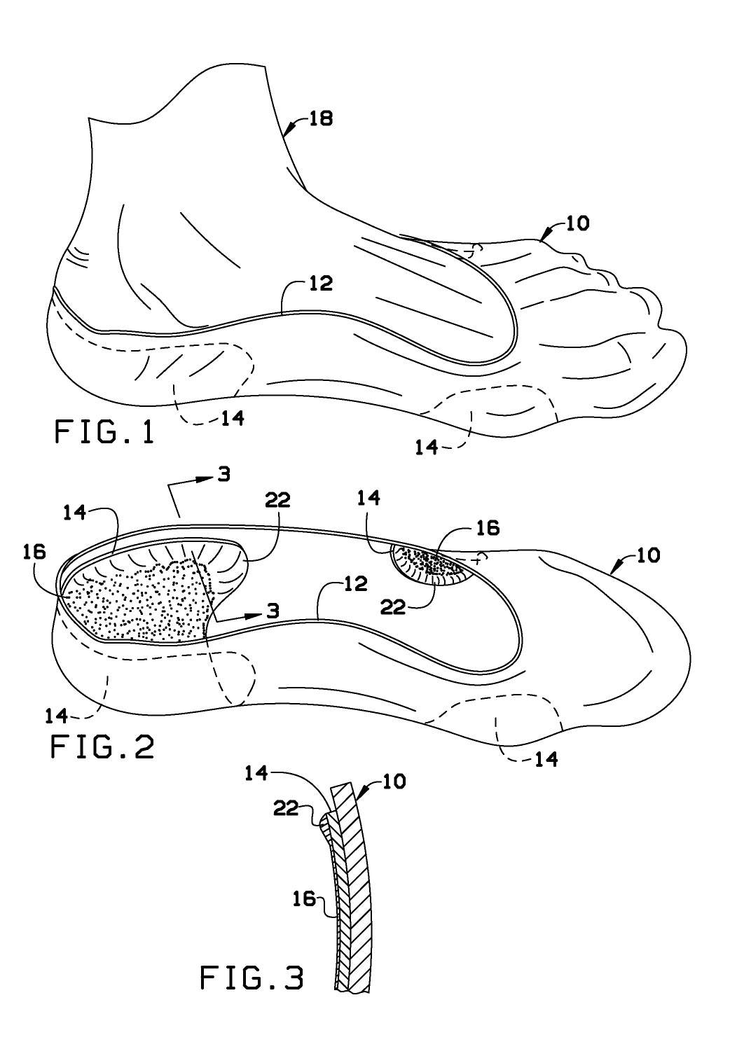

Referring to FIGS. 1 through 5 the present invention may include a garment 10. The garment 10 may include an interior surface, an exterior surface, and an opening 12 where the interior surface and exterior surface meet and where a user may insert their appendage to wear the garment 10. As mentioned above, the garment 10 may fit around an appendage of an animal, such as a human. As illustrated in FIG. 1, the garment 10 may be a foot garment 10 and may fit around an exemplary foot 18 of a user. The garment 10 may be made of a breathable material such as cloth. For example, the cloth may be made of synthetic fibers, such as polyester, or natural fibers, such as cotton. In certain embodiments, the garment 10 may be made of a polymer.

The garment 10 of the present invention may further include at least one insert 14. The insert 14 may be adjacent to the interior surface. For example, the insert 14 may be embedded in the garment 10 or attached to the interior surface of the garment 10. The at least one insert 14 may be made of plastics, vinyl, rubbers or any appropriate polymer. For example, the polymer may include neoprene. In certain embodiments, the thickness of the polymer may be from about 1 millimeter to about 5 millimeters, from about 2 millimeters to about 4 millimeters, or about 3 millimeters.

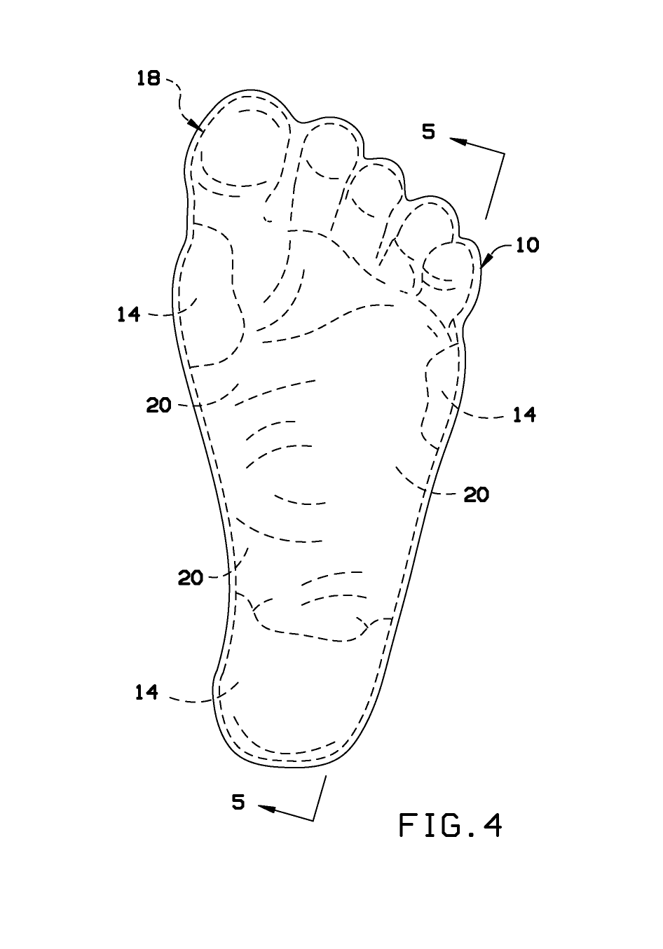

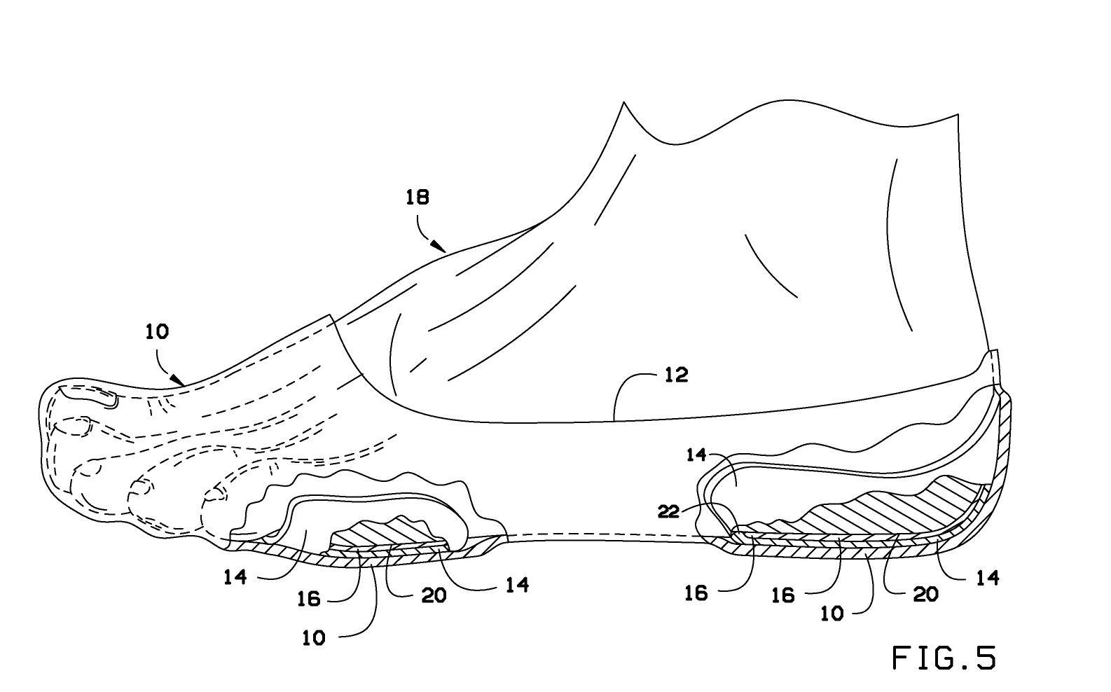

In certain embodiments, the at least one insert 14 may include a plurality of inserts 14 within the garment 10. As illustrated in FIG. 4, the plurality of inserts 14 may be located at the lateral, medial and heel aspects of the garment 10. The inserts 14 of the present invention may include an inner surface that is adjacent to the interior surface of the garment 10, and an outer surface that is in contact with a user's foot 18 when the garment is worn. In certain embodiments, the outer surface of the inserts 14 may include at least one protrusion 22. The protrusion 22 may include an outer parameter rim as illustrated in FIG. 3. The protrusion 22 may further include a plurality of grooves including a plurality of channels in between the grooves.

The present invention may further include a sole 20. The sole 20 of the present invention may include an additional layer on the bottom outer surface of the garment 10. The sole 20 may be a rubber sole 20 to prevent a user from slipping when walking around. For example, the sole 22 may be thin rubber strips that may be applied to the fabric garment 10 at the plantar aspect of the garment to avoid slipping if the wearer uses the garment while walking.

A coating of emollient 16 such as ointments may be applied by a user to the plurality of inserts 14. In certain embodiments, the emollient 16 may be placed in between the protrusions 22, such as in between the parameter or within the channels. The protrusions 22 may provide a reservoir where the emollient 16 may remain in place, for a more focused and concentrated treatment of the skin. The protrusions 22 allow for an effective and site specific treatment. In certain embodiments, protrusions 22 may be from about 0.5 millimeters and to about 2 millimeters in height or from about 1 millimeter to about 1.5 millimeters in height.

The garment 10 may be formed to cover the entire foot 18 or only a portion of the foot 18. For example, the garment 10 may be tapered at the bottom of the foot 18 where only a portion of the sole of the foot 18 is covered. In certain embodiments, the garment 10 allows for the aeration of the central digits. For example, when the garment 10 is used on the foot 18, the garment 10 may leave the second through fourth toes exposed. Such embodiments may further prevent maceration and infections in the exposed locations. Further embodiments of the present invention may also provide for none of the digits being exposed and up to all of the digits being exposed.

A user may wear the fabric garment 10 of the present invention overnight which may facilitate the prevention of occlusion or maceration. The inserts 14 may allow for the emollient 16 to remain in contact with the target areas for treating dry, cracked or fissured feet 18. The inserts 14 may also retain the emollient 16 from leaching out of the garment 10 and staining bedclothes or coming in contact with unnecessary areas of the foot

A method of treating a foot may include the following: providing a medical apparel garment mentioned above; applying an emollient to the outer surface of the at least one polymer insert; and placing the medical apparel on a foot to be treated. The garment may be worn overnight and removed in the morning. In certain embodiments, the present invention is washable, and may be reused again. In certain embodiments, the present invention is disposable and the user may discard the used garment.

It should be understood, of course, that the foregoing relates to exemplary embodiments of the invention and that modifications may be made without departing from the spirit and scope of the invention as set forth in the following claims.

What is claimed is:

1. A medical apparel comprising:
a garment formed to fit on an appendage of an animal, wherein the garment comprises an interior surface and an exterior surface;
at least one polymer insert comprising an outer surface and an inner surface, wherein the inner surface of the polymer insert is adjacent to the interior surface of the garment; and
at least one protrusion protruding from the outer surface of the polymer insert.

2. The medical apparel of claim 1, wherein the at least one protrusion is formed around a parameter of the outer surface of the polymer insert.

3. The medical apparel of claim 1, wherein the at least one protrusion comprises a plurality of protrusions, comprising channels in between the plurality of protrusions.

4. The medical apparel of claim 1, wherein the garment is made of a cloth material.

5. The medical apparel of claim 1, wherein the garment is formed to fit on a foot of a human.

6. The medical apparel of claim 5, wherein the at least one polymer insert comprises at least one of a lateral insert, a medial insert, and a heel insert.

7. The medical apparel of claim 1, wherein the at least one insert is formed to receive an emollient.

8. A medical apparel comprising:
a sock garment comprising an interior surface and an exterior surface, wherein the sock garment is formed to fit on a foot of a human; and
at least one polymer inserts comprising at least one of a lateral insert, a medial insert, and a heel insert, wherein the plurality of inserts is attached to the interior surface of the sock garment.

9. The medical apparel of claim 8, further comprising at least one protrusion protruding from a surface of the polymer insert.

10. The medical apparel of claim 9, wherein the at least one protrusion is formed around a parameter of the surface of the polymer insert.

11. The medical apparel of claim 9, wherein the at least one protrusion comprises a plurality of protrusions, comprising channels in between the plurality of protrusions.

12. A method of treating a foot comprising:
providing a medical apparel comprising:
a garment comprising an interior surface and an exterior surface;
at least one polymer insert comprising an outer surface and an inner surface, wherein the inner surface of the polymer insert is adjacent to the interior surface of the garment; and
at least one protrusion protruding from the outer surface of the polymer insert;
applying an emollient to the outer surface of the at least one polymer insert; and
placing the medical apparel on a foot to be treated.
